(19) United States
(12) Patent Application Publication
(10) Pub. No.: US 2004/0265378 A1
(43) Pub. Date: Dec. 30, 2004

(54) Method and compositions for producing granules containing high concentrations of biologically active substances

(76) Inventors: Yingxu Peng, Memphis, TN (US); Yichun Sun, Germantown, TN (US); James R. Johnson, Germantown, TN (US); Atul J. Shukla, Cordova, TN (US)

Correspondence Address: HOWARD EISENBERG, ESQ., 2206 APPLEWOOD COURT, PERKASIE, PA, 18944, US

(21) Appl. No.: 10/869,180

(22) Filed: Jun. 16, 2004

Related U.S. Application Data
(60) Provisional application No. 60/482,780, filed on Jun. 25, 2003.

(51) Int. Cl. A61K 9/20; A61K 9/14

(52) U.S. Cl. 424/464; 264/109

(57) ABSTRACT

A method for making granules containing a biologically active substance (BAS) whereby the BAS is mixed with a binder to form a powder mixture having a concentration of binder below that which, when the binder is melted, will cause over-wetting of the powder mixture, heating the mixture to a temperature above the melting point of the binder to form an agglomerated powder, and forming granules from the agglomerated powder. The granules may be used to make pharmaceutical dosage forms such as tablets and capsules.

This application claims the benefit of pending U.S. provisional patent application Ser. No. 60/482,780, filed on Jun. 25, 2003.

### FIELD OF THE INVENTION

The invention pertains to the field of granulation processes to produce free flowing granules containing at least one biologically active substance (BAS).

### BACKGROUND OF THE INVENTION

The process of manufacturing pharmaceutical products such as tablets and capsules often requires improving flow and/or compressibility of a powder mixture containing one or more BAS. Tabletting involves compression of blends of powdered materials provided the blends have adequate flow characteristics and compactibility. Powdered materials that have adequate flow characteristics and compactibility may be processed into tablets by a direct compression method. However, if either or both traits, adequate flow and/or adequate compactibility, are missing from the powder blend, then a suitable granulating technique is employed to granulate the powder blend prior to compression so that the resulting granules have improved flow and compactibility.

Powder mixtures are also often granulated if there is a wide variation in particle sizes or bulk densities of the various components of the mixture. By the process of granulation, particle size and bulk density of a powder mixture can become more uniform.

Granulation processes are labor intensive, and are time- and cost-consuming. Therefore, it is desirable to make a tablet containing a BAS and inactive ingredients directly into a tablet without granulation if it is possible to do so. Many BAS powder formulations, especially those containing free-flowing high dose BAS, are capable of being directly compressed into tablets. However, for those BAS having unfavorable flow and/or inadequate compression properties, such as ibuprofen and acetaminophen, granulation prior to compression into tablets is necessary.

The three most commonly used methods of granulation include wet granulation, dry granulation, and hot-melt granulation.

In wet granulation, a BAS is mixed in a mixer with inactive ingredients, such as a filler, binder, and a disintegrant. The resultant powder mixture is then moistened with water, an organic solvent, or an aqueous/organic binder solution which results in a wet granulated mass from which the solvent is then evaporated, typically by drying in an oven, microwave, or an infrared or fluid-bed dryer. The resulting granules can then be compressed into tablets or filled into capsules with or without additional excipients. This method has the disadvantage of requiring an aqueous or organic solvent or binder solution and the application of heat to dry the resultant granules. This process is not suitable for water or heat labile BAS. Moreover, the process requires multiple pieces of equipment and multiple steps, and requires the expenditure of significant quantities of energy. Organic solvents are not often used, due to environmental concerns and danger of explosions.

Dry granulation is achieved typically by either a slugging or a roller compaction process. With slugging, a BAS is mixed with inactive ingredients and is compressed into slugs, large tablets about 1 inch in diameter. The tablets are then broken and sieved through appropriate sieves to obtain granules of the desired size. The sieved granules are then compressed into tablets or filled into capsules with or without additional excipients.

With roller compaction, a BAS is mixed with inactive ingredients and the mixture is passed through rollers to form a compacted sheet of the material. The compacted sheet is then passed through a comminuting mill fitted with an appropriate size of sieve in order to obtain granules of the desired size. The resulting granules are then compressed into tablets or filled into capsules with or without additional excipients.

Dry granulation methods have several disadvantages. They require additional equipment. Moreover, with these methods it is often difficult to control the size of the resultant granules and loss of starting material is usually greater with dry granulation than with other methods. The dry granulation process also produces significant amounts of dust, which represents loss of materials and may cause a hazard to equipment and personnel.

Hot-melt granulation utilizes a material referred to as a hot-melt binder, which is a solid or semi-solid at room temperature and which melts at a temperature below that at which the BAS of interest melts. Typically, the binder melts at a temperature between 30° C. and 200° C. A solvent such as water or an organic compound is not necessary to initiate binding in this method.

The low-melting binder, when heated to a sufficiently high temperature, liquifies or becomes tacky. This tacky and/or liquified binder spreads itself over the surface of the powdered or particulate matter in a mixture and forms agglomerates of the mixture, which upon cooling, forms a solid granulated mass in which the powder or particulate starting materials are bound. The resultant granules can then be provided to a tablet press, mold, or encapsulator, such as a capsule filling machine, for preparing the desired dosage form with or without additional excipients.

Hot-melt granulation utilizes particular equipment, such as rotating pan, extruder, fluidized bed granulator, low shear mixer, and high shear mixer granulator. The energy to melt the binder may come from heat dissipated from circulating hot liquid, such as water or oil, steam, hot air, or friction such as due to the equipment used in hot-melt granulation.

Hot-melt techniques eliminate the disadvantages present with wet and dry granulation techniques. Additional solvents and extensive drying times associated with wet granulation methods are eliminated as are the dust and loss problems associated with dry granulation methods. Moreover, hot-melt techniques permit the production of denser granules in a shorter time period than is possible with other granulation methods.

Because of its advantages, hot-melt granulation techniques have been extensively utilized and several adaptations of this technique have been made. Several patents disclose the use of hot-melt and similar granulation techniques to produce or to modify granules for immediate release and delayed release pharmaceutical compositions. The patents include Speiser, U.S. Pat. No. 4,013,784; Blichare, U.S. Pat. No. 4,132,753; Ahrens, U.S. Pat. No. 4,935,246; Royce, U.S. Pat. No. 5,403,593; Kristensen, U.S. Pat. No. 5,476,667; Hurner, U.S. Pat. No. 5,667,807; and Heafield, U.S. Pat. No. 6,143,328, each of which is incorporated herein by reference. Most notable of these patents are those of Blichare, Royce, Kristensen, and Hurner.

Blichare discloses contacting a wax-like material with a powdered medicament at a temperature above the melting point of the wax-like material. This results in the powdered medicament sinking into the molten surface of the wax-like pieces to form spherical granules having an interior of a medicament surrounded by a coating of the wax-like material. Such granules suitable for time-release dosage containing up to about 80% active therapeutic ingredient were reportedly obtained by Blichare.

Royce discloses a hot-melt granulation technique utilizing 5 to 90% concentration of a hydrophilic cellulose ether polymer, 5 to 50% of a granulating medium (binder), and a therapeutically active medicament. A mixture containing these components, plus additional excipients, is heated for a time sufficient to completely liquefy the mixture, which is then cooled to room temperature and formed into granules.

Kristensen discloses a two-step process by which granules containing high concentrations of BAS may be obtained. A BAS in a cohesive form, such as having a mean particle size less than 30 microns, is mixed with a binder. The mixture is heated to melt the binder and form overwetted spherical pellets. Additional quantities of BAS is then added to the overwetted spherical pellets to obtain the desired pellets, which may have a ratio of BAS:binder as high as 95:5.

Hurner discloses a hot-melt granulation method whereby an active compound having a melting point between 30 and 200° C. fulfils the function of a binder. According to the method of Hurner, a low-melting active compound and inactive compounds such as binders, fillers, and disintegrants, are mixed and heated to a temperature at which a part of the active compound itself is melted. Granules are formed by extrusion, a process which requires over-wetting. Thus, the low-melting active compound acts as the binder according to the invention of Hurner.

Among other disadvantages, the hot-melt process of each of the above patents requires over-wetting. “Over-wetting” is described in Kristensen as being well known to one skilled in the art and implying the addition of excess binder such as to cause the surface of the granules to become tacky. Such over-wetting is disadvantageous because it necessitates the use of a high concentration of a low-melting binder and additional heat and time to liquefy the binder so as to form tacky granules. Thus, such processes involving over-wetting limit the concentration of BAS that can be present in the granules.

### BRIEF DESCRIPTION OF THE FIGURES

FIG. 1 is a bar graph that shows the particle size distribution of ibuprofen granules obtained by the method of the invention.

FIG. 2 is a graph shows the effect of compression force on the dissolution of ibuprofen from tablets containing 90% ibuprofen and 4% of a binder (PEG 8000) produced in accordance with the method of the invention.

FIG. 3 is a bar graph that shows the particle size distribution of acetaminophen granules obtained by the method of the invention.

FIG. 4 is a graph shows the effect of compression force on the hardness of tablets containing 90% acetaminophen and 7.5% of a binder (PEG 8000) produced in accordance with the method of the invention.

FIG. 5 is a graph shows the effect of compression force on the dissolution of acetaminophen from tablets containing 90% acetaminophen and 7.5% of a binder (PEG 8000) produced in accordance with the method of the invention.

FIG. 6 is a bar graph that shows the particle size distribution of aspirin granules obtained by the method of the invention.

FIG. 7 is a graph shows the effect of compression force on the hardness of tablets containing 90% aspirin and 6% of a binder (PEG 8000) produced in accordance with the method of the invention.

### DETAILED DESCRIPTION OF THE INVENTION

The inventors have discovered a method by which free-flowing compressible granules containing one or more biologically active substances may be obtained and by which several disadvantages present with prior art methods are overcome. The method of the invention is a hot-melt granulation method, according to which method a fine powder containing a biologically active substance is agglomerated using a melting binder which is solid at room temperature but which melts when subjected to a temperature above the melting point of the binder. In accordance with the method of the invention, the temperature is preferably below the melting point of the biologically active substance. Preferably, the melting point of the binder is between 30° C. to 200° C. The hot-melt granulation can be carried out using equipment, such as rotating pan, extruder, fluidized bed granulator, low shear mixer, and high shear mixer granulator.

In accordance with the method of the invention, a powder containing a biologically active substance is mixed with a binder and this mixture is caused to be heated to a temperature above the melting point of the binder so that the binder melts, wherein the concentration of the binder in the mixture is at or above that which will agglomerate the powder but below that at which will result in over-wetting of the mixture upon the melting of the binder. All concentrations used herein are w/w, unless indicated otherwise. The heat supplied to the mixture may be from any source, such as from an external source like circulating hot liquid (such as water or oil), hot air or steam, or microwave, infrared sources, or heating tape. Preferably, energy released from high-shear mixing due to friction heats the binder to its melting point. Following or during the process of agglomeration, granules are formed, which are cooled to ambient temperature. The granules may be encapsulated into capsules or compressed into tablets, with or without adding excipients such as a filler, disintegrant, glidant, and/or lubricant.

Preferably, the granules are formed without subjecting the mixture to external pressure, such as required for an extrusion process. However, if desired, such pressure may be utilized and granules from the mixture may be formed by an extrusion process. This method is not preferred because typically extrusion requires over-wetting of the granule-forming mixture. However, extrusion-based processes which are suitable for the invention are those that do not utilize over-wetting of the granule forming mixture.

Preferably, but not necessarily, the concentration of the binder in the mixture is less than 5%. However, concentrations higher than 5% may be used, so long as the concentration of the binder is below that at which over-wetting will occur when the binder is melted.

Any concentration of BAS may be combined with the binder to form the granules. However, because the method of the invention is capable of providing granules with very high concentrations of BAS, it is preferred that the concentration of BAS in the mixture be at least 80%, with the remainder of the mixture being composed of binder and other excipients. More preferably, the concentration of the BAS in the mixture is at least 85% and most preferably at least 90%. If desired, the concentration of BAS in the mixture may be as high as 95% or even higher. Maximum concentration of BAS in the mixture will depend upon several factors, including the properties of the BAS and of the binder, the size and specific surface area of the particles of the BAS, the concentration of binder in the mixture, and the presence of additional excipients in the mixture.

If additional excipients, such as (a) fillers like lactose, microcrystalline cellulose, starch or calcium phosphate salts, (b) disintegrants like cross-linked carboxymethylcellulose such as sold under the brand name AC-DI-SOL® (FMC Corporation, Philadelphia, Pa.), sodium starch glycolate such as sold under the brand name ExploTab® (J. Rettenmaier USA, Schoolcraft, Mich.), or cross-linked polyvinyl pyrrolidone such as crospovidone, (c) glidants such as silicon dioxide or talc, (d) flavoring agents, (e) coloring agents, (f) dry binders such as polyvinyl pyrrolidone, hydroxypropylmethylcellulose, a binder of hydrophilic or hydrophobic polymer or waxy material such as hydroxypropyl methylcellulose, hydroxypropylcellulose, hydroxyethylcellulose, sodium carboxymethylcellulose (Na-CMC), methylcellulose, microcrystalline cellulose, ethylcellulose, carnauba wax, or stearyl alcohol, or one or more of the hydrophilic cellulose ether polymers disclosed in Royce, U.S. Pat. No. 5,403,593 (incorporated herein by reference), or (g) lubricants such as stearic acid, magnesium or calcium stearate, or hydrogenated vegetable oils, are desired, these may be added extra-granularly or intra-granularly. The release characteristics of the BAS from the resulting granules, capsules, or compressed tablets may be modulated by the physicochemical properties of the excipients that may be added either intragranularly or extragranularly.

If such additional excipients, such as the hydrophilic cellulose ether polymer (HCEP) disclosed in the Royce patent, is utilized, it is preferred that the concentration of the additional excipient in the mixture and in the granules be less than 5%. However, the concentration of the additional excipient may be 5% or higher, if desired.

The melt binder may be any binder known in the art, or later discovered, that is solid or semisolid at ambient temperature, but can be melted at a temperature between 30° C. and 200° C., and which is used to formulate granules for the production of pharmaceutical formulations. Examples of suitable binders include those disclosed in U.S. Pat. No. 5,403,593, which is incorporated herein in its entirety by reference, a lipid or waxy component, a sugar, a poloxamer, or a polymer of ethylene glycol.

A suitable lipid component use in the invention is one having a melting point of about 30° C. to about 200° C. The term “lipid component” refers to lipid and lipid-like materials, including lecithin, fatty esters, fatty acids and salts thereof, fatty alcohols, fatty amines, fatty amides, glycerides, glycolipids, steroids, natural and synthetic waxes, and mixtures thereof. Examples of fatty acid esters which may also be used as a hot-melt binder include mono-, di- or triesters of polyglycerols with fatty acids. The polyglycerol includes but is not limited to diglycerol, triglycerol, tetraglycerol, pentaglycerol, hexaglycerol, heptaglycerol, octaglycerol, nonaglycerol, decaglycerol, pentadecaglycerol, eicosaglycerol, and triacontaglycerol. The fatty acid includes but is not limited to saturated or unsaturated fatty acids each containing about 8 to about 40, preferably about 12 to about 28, and more preferably about 15 to about 22 carbon atoms. Examples of the fatty acid include stearic acid, oleic acid, lauric acid, linoleic acid, linolenic acid, ricinoleic acid, caprylic acid, capric acid, palmitic acid, and behenic acid, or salts, such as sodium or potassium salts, thereof. The polyglycerol fatty acid ester includes but is not limited to behenic acid hexa(tetra)glyceride, caprylic acid mono(deca)glyceride, caprylic acid di(tri)glyceride, capric acid di(tri)glyceride, lauric acid mono(tetra)glyceride, lauric acid mono (hexa)glyceride, lauric acid mono(deca)glyceride, oleic acid mono(tetra)glyceride, oleic acid mono(hexa)glyceride, oleic acid mono(deca)glyceride, oleic acid di(tri)glyceride, oleic acid di(tetra)glyceride, oleic acid sesqui(deca)glyceride, oleic acid penta(tetra)glyceride, oleic acid penta(hexa)glyceride, oleic acid deca(deca)glyceride, linoleic acid mono(hepta)glyceride, linoleic acid di(tri)glyceride, linoleic acid di(tetra) glyceride, linoleic acid di(hexa)glyceride, stearic acid mono(di)glyceride, stearic acid mono(tetra)glyceride, stearic acid penta(tetra)glyceride, stearic acid mono(deca)glyceride, stearic acid tri(tetra)glyceride, stearic acid penta(hexa) glyceride, stearic acid tri(hexa)glyceride, stearic acid deca(deca) glyceride, palmitic acid mono(tetra)glyceride, palmitic acid mono(hexa)glyceride, palmitic acid mono(deca)glyceride, palmitic acid tri(tetra)glyceride, palmitic acid tri(hexa)glyceride, palmitic acid sesqui(hexa)glyceride, palmitic acid penta(tetra)glyceride, palmitic acid penta(hexa)glyceride, palmitic acid deca(deca)glyceride, and polyglycerol polyricinolate (e.g. tetraglycerol polyricinolate, etc.). Other suitable glyceryl esters of fatty acids include triglyceryl ester, glyceryl distearate, glyceryl tristearate, glyceryl monostearate, glyceryl dipalmitate, glyceryl tripalmitate, glyceryl monolaurate, glyceryl dodecosanoate, glyceryl tridecosanoate, glyceryl monodecosanoate, glyceryl monocaprate, glyceryl dicaprate, glyceryl tricaprate, glyceryl monomyristate, glyceryl dimyristate, glyceryl trimyristate, glyceryl monodecanoate, glyceryl didecosanoate, glyceryl tridecosanoate.

Examples of suitable fatty alcohols include higher alcohols of about 16 to about 22 carbon atoms, such as cetyl alcohol and stearyl alcohol; fatty acid glycerol esters such as the monoglycerides, diglycerides, or triglycerides of the above-mentioned fatty acids; hydrogenated oils such as hydrogenated cottonseed oil, hydrogenated castor oil, hydrogenated soybean oil, and hydrogenated tallow.

Waxes such as beeswax, carnauba wax, sperm wax, and castor wax, hydrocarbons such as paraffin, microcrystalline wax, and wool wax, and other waxes which are solid at room temperature or mixtures thereof may also be used.

Examples of sugars which may be used as a hot-melt binder include, but are not limited to, fructose, dextrose, xylitol, sorbitol, maltitol, and polydextrose. Examples of poloxamers which may be used as a hot-melt binder, but are not limited to, poloxamer 188, 237, 338, and 407.

Another suitable binder is polyethylene glycol (PEG) which has the formula HO—(CH₂CH₂O)ₙ—H, wherein n represents the average number of oxyethylene groups. PEG is generally designated according to the average molecular weight of the polymer. Preferred PEGs are PEG 1000, 1450, 1540, 2000, 3000, 3350, 4000, 4600, 6000, 8000, and PEG 20000, which have a melting point ranging from about 37° C. to about 65° C.

The method of the invention can be used to produce granules containing a high concentration of BAS. The method of the invention is especially useful for producing granules that are suitable either for compressing into tablets or encapsulating into capsules wherein the BAS that is present in the granules is unsuitable for processing into tablets or capsules due to its unfavorable physicochemical properties, such as flowability, density, particle size, or compactibility. Such BAS, referred to herein as “non-compressible BAS”, include but are not limited to ibuprofen, acetaminophen, aspirin, and naproxen. Other non-compressible BAS include but are not limited to antibiotics, ketoprofen, indomethacin, ranitidine, sucralfate, vitamin C, probucol, nicotinic acid, aminocaproic acid, pentoxyfylline, quinidine gluconate, nifedipine, verapamil hydrochloride, cholestyramine, metoproplol tartrate, tocainamide hydrochloride, ethotoin, phenacemide, and carbidopa.

It has been unexpectedly discovered that, by the method of the invention, granules that are suitable for compression into tablets may be obtained even when such granules contain a compressible or non-compressible BAS. Such compressible granules are obtained by the method of the invention even when the concentration of the BAS in the granule is 80% or higher, when the concentration of the BAS is 85% or higher, when the concentration of the BAS is 90% or higher, and even when the concentration of the BAS is 95% or higher.

If desired, the granules produced by the method of the invention may be compressed into tablets or encapsulated into capsules, with or without additional excipients. Such granules with a high concentration of BAS are particularly useful for preparing tablets or capsules containing high dose drugs, since additional excipients that may be added to granules prior to compression or encapsulation would increase the size of the finished dosage form and make it difficult to be administered to patients. Additional excipients such as directly compressible fillers, binders, disintegrants such as cross-linked sodium carboxymethylglucose, glidants, and lubricants may be added if desired and a mixture containing these ingredients may be made into a finished dosage form, such as by compression into tablets or filling into capsules. The final concentration of the BAS in the finished dosage form could range from 1 to 95%. However, it is preferable to have a concentration of BAS between 5 and 90% in the finished dosage form. More preferably, the concentration of BAS in the tablets or capsules is 80% or higher, even more preferably 85% or higher, most preferably 90% or higher, and may even have a BAS concentration of 95% or higher.

In another embodiment, the invention is a BAS-containing granule that is directly compressible to a tablet. The granule is formed by the method of the invention from a mixture containing the BAS and a binder. The granule of the invention contains the binder at a concentration at or above that which provides complete agglomeration of the BAS in the mixture and below that which results in over-wetting of the mixture. The granules may contain an additional excipient, as described above, preferably at a concentration of 0% to less than 5%. The concentration of the additional excipient may be 5% or higher, if desired.

The BAS in the granule may be a compressible BAS or a non-compressible BAS. Concentration of the BAS in the granule may be 80% or higher, such as 85% or higher. Preferably, the concentration of the BAS in the granule is 90% or higher, and most preferably 95% or higher.

In another embodiment, the invention is a compressible or processable granule containing a binder and any one or more non-compressible BAS. The BAS in the granule may be selected from the group consisting of ibuprofen, acetaminophen, aspirin, naproxen, antibiotics, ketoprofen, indomethacin, ranitidine, sucralfate, vitamin C, probucol, nicotinic acid, aminocaproic acid, pentoxyfylline, quinidine gluconate, nifedipine, verapamil hydrochloride, cholestyramine, metoproplol tartrate, tocainamide hydrochloride, ethotoin, phenacemide, and carbidopa, wherein the total concentration of BAS in the granule is 80% or higher, such as 85% or higher. Preferably, the concentration of BAS in the granule is 90% or higher, and most preferably 95% or higher.

Preferably, according to this embodiment, the granule contains a binder at a concentration below that which is sufficient to cause over-wetting of the granule. Additional excipients, such as fillers, disintegrants, glidants, flavoring agents, coloring agents, dry binders, or lubricants, may be added extra-granularly or intra-granularly as desired for preparing finished dosage forms.

In another embodiment, the invention is a compressed tablet containing any one or a combination of BAS selected from the group consisting of ibuprofen, acetaminophen, aspirin, and naproxen wherein the concentration of the BAS in the tablet is 80% or higher, preferably 85% or higher, more preferably 90% or higher, and most preferably 95% or higher. The tablet of the invention may further include additional excipients such as extra-granular or intra-granular fillers, disintegrants, glidants, flavoring agents, coloring agents, dry binders, or lubricants. The concentration of binder in the tablet is preferably less than that which, when combined in a mixture with the BAS in the tablet, would cause over-wetting of such a mixture of binder and BAS during a hot melt granulation process. As described above, the concentration of an additional excipient in the tablet is preferably 0% to less than 5%. The concentration of the additional excipient may be 5% or higher, if desired.

The invention is further described in the following non-limiting examples.

### EXAMPLE 1

### Ibuprofen

TABLE 1
Composition of Ibuprofen tablets

| | | Concentration | Weight Per | Total Batch |
|---|---|---|---|---|
| Material | Ingredients | in Tablets (%) | Tablet (mg) | Size (g) |
| Granules | PEG 8000 | 4 | 8.88 | 525.2 |
| | Ac-Di-Sol ® | 1.5 | 3.33 | |
| | Ibuprofen | 90 | 200 | |
| Extra- | Ac-Di-Sol ® | 1.5 | 3.33 | 8.3 |
| Granular | | | | |
| Disintegrant | | | | |
| Glidant | Cab-O-Sil ® | 1 | 2.22 | 5.5 |
| Lubricant | Stearic acid | 2 | 4.44 | 11.0 |
| Total | | 100 | 222 | 550 |

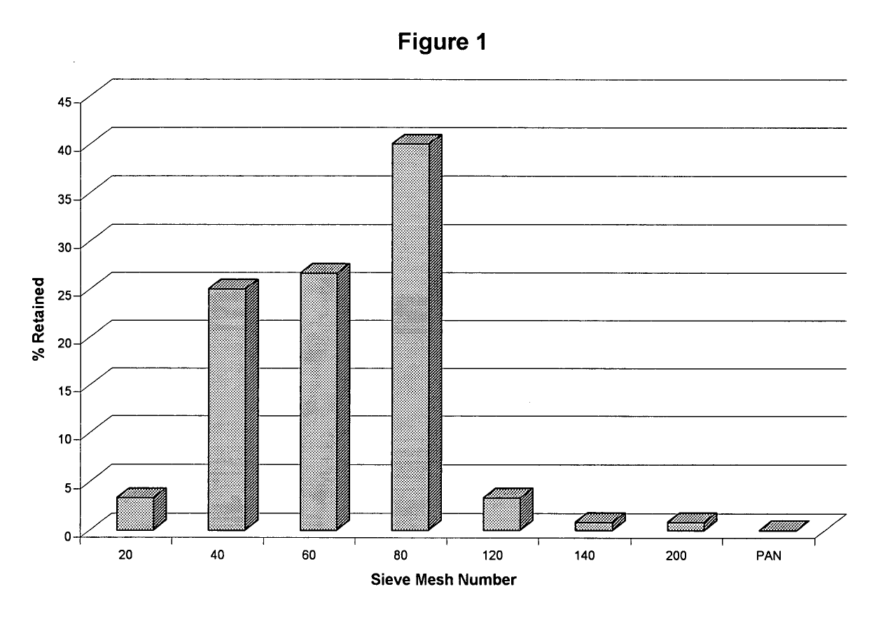

In accordance with the method of the invention, granules containing ibuprofen at a concentration of 94.2% and tablets containing ibuprofen at a concentration of 90% w/w were produced as follows. Appropriate quantities of ibuprofen, a binder (PEG 8000), and a disintegrant (Ac-Di-Sol®), according to concentrations indicated in Table 1 were weighed and loaded into the jacketed bowl of a high-shear mixer granulator (Model: 3 VG, Robot Coupe USA, Inc., Jackson, Miss.). The granulator was operated for 2 minutes at 1500 rpm in the forward mode to blend the materials. After blending, the granulator was operated at 2000 rpm in the reverse mode until the granulation end-point was reached. The product temperature was approximately 45-50° C., which was much lower than the melting point of ibuprofen (78° C.). The granulation time was approximately 6 minutes. The granulation end-point was determined using Scope View software (Radio Shack, Fort Worth, Tex.), which measures the current of the motor of the granulator, and by visual observation. The granules were removed from the bowl and allowed to cool to ambient temperature. Particle size distribution of the granules was determined by sieve analysis and is shown in FIG. 1.

Compressed tablets were produced from the granules. As indicated in Table 1, appropriate quantities of extra-granular disintegrant Ac-Di-Sol® and a glidant, Cab-O-Sil® (Cabot Corporation, Tuscola, Ala., USA), which was sieved through a # 60 mesh sieve, were blended with the obtained granules in a V-shell blender for 3 minutes. Appropriate quantities of sieved stearic acid was added to the mixture in the V-shell blender and mixed for an additional 2 minutes. The resulting mixture was compressed into tablets using ⅜ standard concave tablets tooling on a rotary tablet press (Model: HT-AP 18 SS-U/I, Elizabeth Hata International, Inc., North Huntingdon, Pa.).

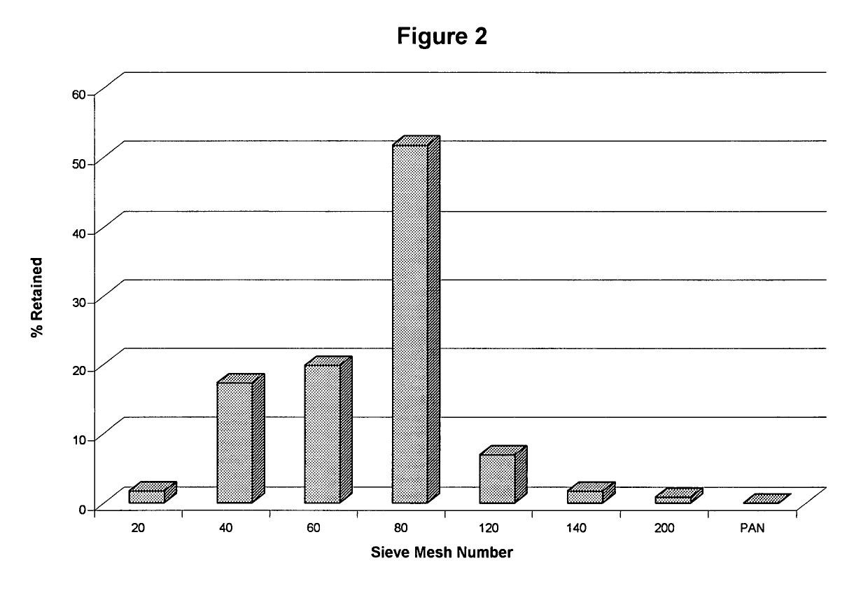

Quality control tests such as weight variation, thickness, hardness and dissolution were performed on the obtained tablets. The results of weight variation, thickness, and hardness tests are shown in Table 5 and the dissolution profile of the ibuprofen from the tablets is shown in FIG. 2.

### EXAMPLE 2

### Acetaminophen

TABLE 2
Composition of Acetaminophen tablets

| | | | | Total |
|---|---|---|---|---|
| | | Concentration | Weight Per | Batch |
| Material | Ingredients | in Tablets (%) | Tablet (mg) | Size (g) |
| Granules | PEG 8000 | 7.5 | 8.88 | 287.10 |
| | Ac-Di-Sol ® | 1.0 | 3.33 | |
| | Acetaminophen | 90 | 500 | |
| Extra- | Ac-Di-Sol ® | 1.0 | 3.33 | 2.92 |
| Granular | | | | |
| Disintegrant | | | | |
| Lubricant | Magnesium | 0.5 | 4.44 | 1.46 |
| | Stearate | | | |
| Total | | 100 | 555.6 | 291.48 |

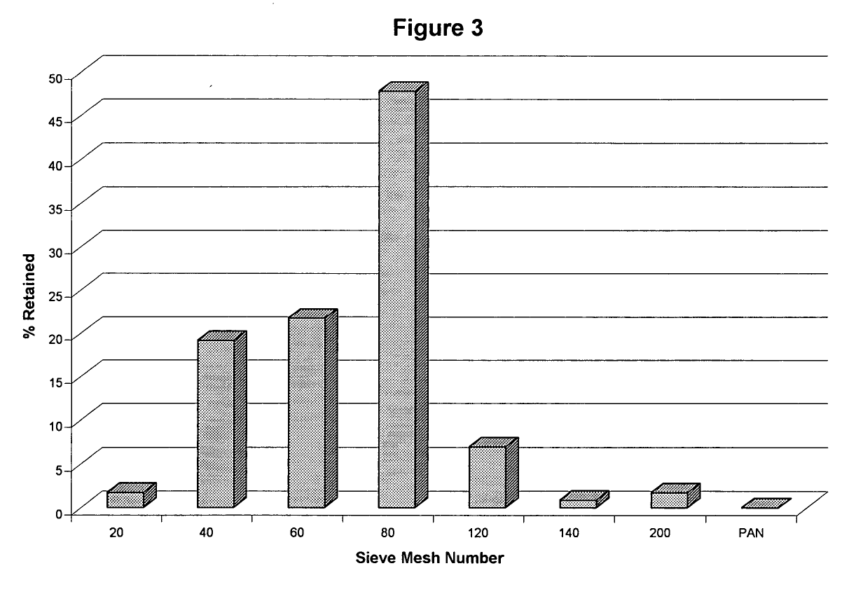

In accordance with the method of the invention, granules containing acetaminophen at a concentration of 91.4% and tablets containing acetaminophen at a concentration of 90% w/w were produced as follows. As indicated in Table 2, appropriate quantities of a binder (PEG 8000), a disintegrant (Ac-Di-Sol®) and acetaminophen were weighed and loaded into the jacketed bowl of the high-shear mixer granulator (Model: 3 VG, Robot Coupe USA, Inc., Jackson, Miss.). The granulator was operated for 2 minutes at 1500 rpm in the forward mode to blend the materials. After blending, the granulator was operated at 2000 rpm in the reverse mode until the granulation end-point was reached (approximately 8 minutes), which was determined using Scope View software (Radio Shack, Fort Worth, Tex.) and by visual observation. The granules were removed from the bowl and allowed to cool to ambient temperature. Particle size distribution of the granules was determined by sieve analysis, as shown in FIG. 3.

Tablets were compressed from the obtained granules, as follows. As shown in Table 2, an appropriate quantity of extra-granular disintegrant Ac-Di-Sol® was added to the granules and blended in a V-shell blender for 3 minutes. As indicated in Table 2, an appropriate quantity of a lubricant, magnesium stearate was added to the mixture in the V-shell blender and mixed for an additional 2 minutes. The resulting blended mixture was compressed into tablets using 7/16 standard concave tablet tooling on a rotary tablet press (Model: HT-AP 18 SS-U/I, Elizabeth Hata International, Inc., North Huntingdon, Pa.).

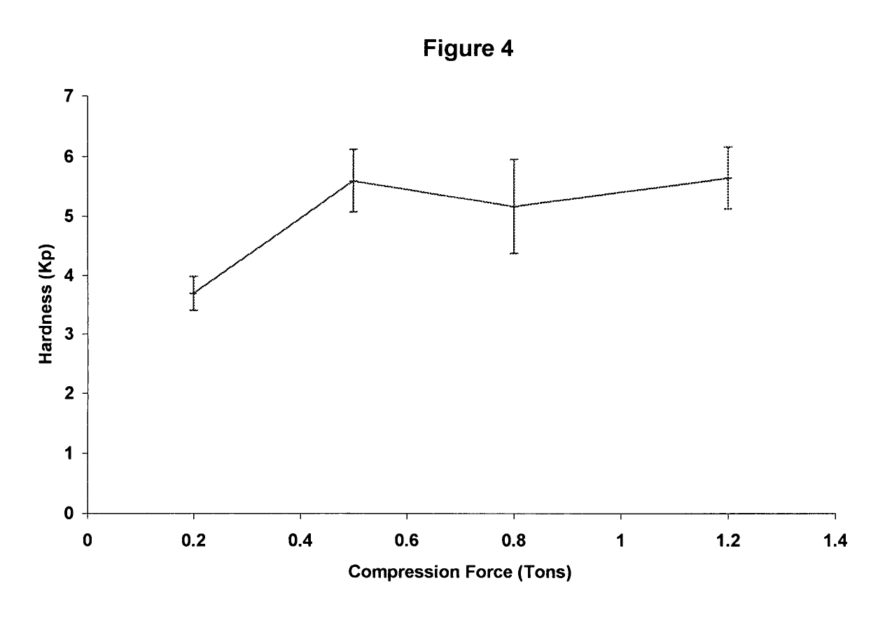

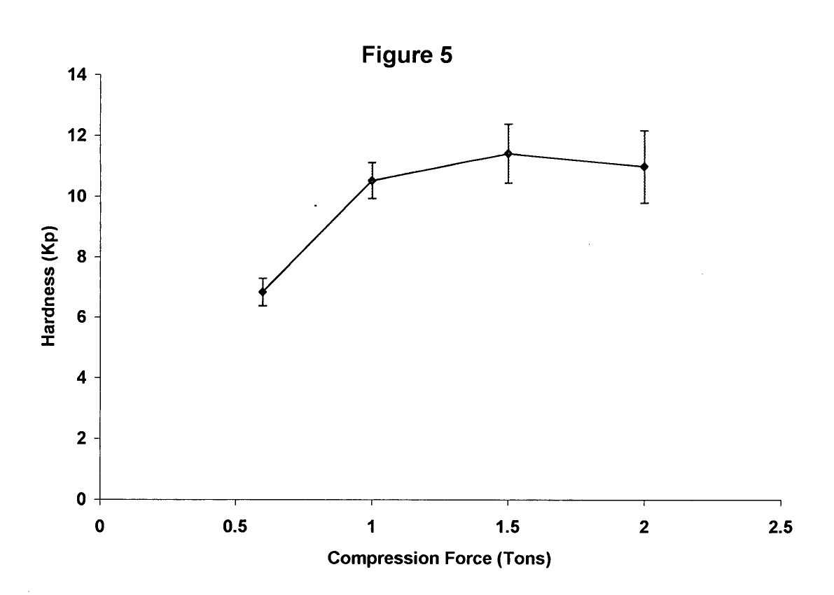

Tableting parameters such as fill-depth, pre-compression force, main compression force, turret speed, and ejection force were recorded. The compression profile of the acetaminophen tablets is shown in FIG. 4. Quality control tests such as weight variation, thickness, hardness, and dissolution were performed on the obtained tablets. The results of weight variation, thickness, and hardness tests are shown in Table 5 and the dissolution profile of the drug from the tablets is shown in FIG. 5.

### EXAMPLE 3

### Aspirin

TABLE 3
Composition of Aspirin tablets

| | | Concentration | Weight Per | Total Batch |
|---|---|---|---|---|
| Material | Ingredients | in Tablets (%) | Tablet (mg) | Size (g) |
| Granules | PEG 8000 | 6 | 8.88 | 335.34 |
| | Ac-Di-Sol ® | 1.5 | 3.33 | |
| | Aspirin | 90 | 500 | |
| Extra- | Ac-Di-Sol ® | 1.5 | 3.33 | 5.16 |
| Granular | | | | |
| Disintegrant | | | | |
| Lubricant | Stearic acid | 1 | 4.44 | 3.44 |
| Total | | 100 | 555.6 | 343.94 |

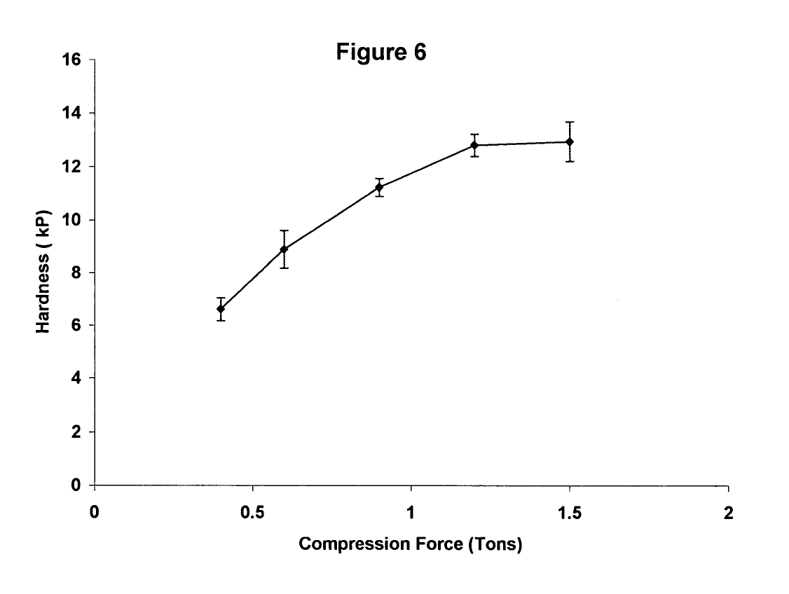

In accordance with the method of the invention, granules containing aspirin (acetylsalicylic acid) at a concentration of 92.3% and tablets containing aspirin at a concentration of 90% w/w were produced as follows. As indicated in Table 3, appropriate quantities of a binder (PEG 8000), a disintegrant (Ac-Di-Sol®) and aspirin were weighed and loaded into the jacketed bowl of the high-shear mixer granulator (Model: 3 VG, Robot Coupe USA, Inc., Jackson, Miss.). The granulator was operated for 2 minutes at 1500 rpm in the forward mode to blend the materials. After blending, the granulator was operated at 2000 rpm in the reverse mode until the granulation end-point was reached (approximately 6 minutes), which was determined using Scope View software (Radio Shack, Fort Worth, Tex.) and by visual observation. The granules were removed from the bowl and allowed to cool to ambient temperature. Particle size distribution of the granules was determined by sieve analysis and is shown in FIG. 6.

Tablets were compressed from the obtained granules. An appropriate quantity of extra-granular disintegrant (Ac-Di-Sol®) was added the to granules and was blended in a V-shell blender for 3 minutes. Appropriate quantities of sieved stearic acid was added to the mixture in the V-shell blender and mixed for an additional 2 minutes. The resulting blended mixture was compressed into tablets using ⅜ standard concave tablet tooling on a rotary tablet press (Model: HT-AP 18 SS-U/I, Elizabeth Data International, Inc., North Huntingdon, Pa.).

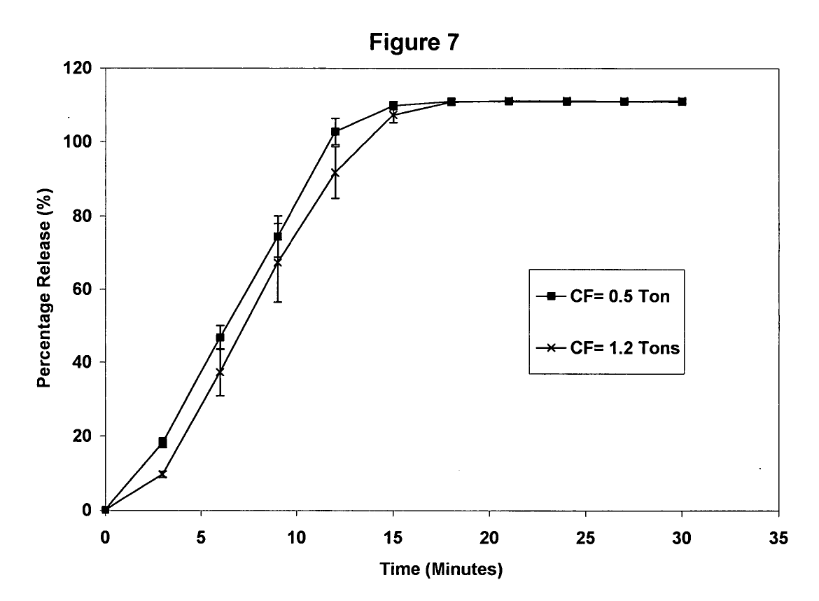

Tableting parameters such as fill-depth, pre-compression force, main compression force, turret speed, and ejection force were noted. The compression profile of the aspirin tablets is shown in FIG. 7. Quality control tests such as weight variation, thickness, and hardness were performed on the obtained tablets, and the results are shown in Table 5.

### EXAMPLE 4

### Characterization of the Granules of Examples 1 to 3

The particle size distributions of the granules produced in Examples 1 to 3 are shown in FIGS. 1, 3, and 6, respectively. The average particle size and size distribution characteristics of the particles were further analyzed and are shown in Table 4.

TABLE 4
Characterization of the granules

| | |
|---|---|
| API in the | Particle Size |

| Granules | d_{g} | σ_{g} |
|---|---|---|
| Ibuprofen | 429.7 | 1.83 |
| Acetaminophen | 368.5 | 1.90 |
| Aspirin | 379.0 | 1.89 |

d_{g}: geometric average particle size
σ_{g}: standard deviation of particle size distribution

### EXAMPLE 5

### Weight Variation, Thickness, and Hardness of the Tablets of Examples 1 to 3

TABLE 5
Weight variation, thickness and hardness of tablets
compressed from granules containing a high
concentration of BAS

| | CF | Weight | RSD | Thickness | Hardness |
|---|---|---|---|---|---|
| Tablets | (mTon) | (mg) | (%) | (mm) | (kP) |
| Ibuprofen | 0.2 | 223.88 | 0.73 | 3.9688 | 3.69 |
| (Example 1) | 0.5 | 224.24 | 0.75 | 3.8324 | 5.59 |
| | 0.8 | 224.44 | 0.74 | 3.8184 | 5.16 |
| | 1.2 | 224.01 | 0.75 | 3.8030 | 5.64 |
| Acetaminophen | 0.6 | 557.14 | 0.53 | 5.9426 | 6.83 |
| (Example 2) | 1.0 | 565.00 | 0.58 | 5.8854 | 10.52 |
| | 1.5 | 547.61 | 0.28 | 5.6654 | 11.41 |
| | 2.0 | 550.79 | 0.55 | 5.6514 | 10.98 |
| Aspirin | 0.4 | 552.46 | 0.75 | 5.5804 | 6.6 |
| (Example 3) | 0.6 | 555.58 | 0.81 | 5.4544 | 8.88 |
| | 0.9 | 555.6 | 0.18 | 5.3916 | 11.22 |
| | 1.2 | 562.96 | 0.72 | 5.4496 | 12.8 |
| | 1.5 | 565.12 | 0.20 | 5.418 | 12.94 |

CF: compression force
mTon: metric ton
RSD: relative standard deviation
kP: kiloponds

### EXAMPLE 6

### Prior Art Methods to Produce Acetaminophen Granules

Granules containing acetaminophen were attempted to be made according to the method of Royce, U.S. Pat. No. 5,403,593, utilizing concentrations of components in two different compositions as shown in Table 6.

TABLE 6
Composition of acetaminophen granules prepared according to
Royce, U.S. Pat. No. 5,403,593

| | COMPOSITION | COMPOSITION |
|---|---|---|
| COMPONENT | #1 | #2 |
| hydroxypropyl methylcellulose | 5% | 7.5% |
| (HPMC K15M) (Dow | | |
| Chemical Co., Midland, MI, | | |
| USA) | | |
| PEG 8000 | 2.5% | 3.75% |
| glyceryl palmitostearate | 2.5% | 3.75% |
| (Precirol ATO-5) (Gattefosse | | |
| Corp., Elmsford, NY, USA) | | |
| acetaminophen (APAP) | 90% | 85% |

Utilizing the prior art method disclosed in Royce with the above compositions, free flowing granules were not obtained.

Further modifications, uses, and applications of the invention described herein will be apparent to those skilled in the art. It is intended that such modifications be encompassed in the following claims.

1. A method for preparing granules containing one or more biologically active substances (BAS) comprising mixing a binder with the BAS to obtain a powder mixture, heating the powder mixture to a temperature above the melting point of the binder to obtain an agglomerated powder, wherein the concentration of the binder in the powder mixture is at least that which will agglomerate the powder mixture when the binder is melted and is below that which will result in over-wetting of the powder mixture when the binder is melted, and permitting granules to form from the agglomerated powder.

2. The method of claim 1 wherein the heating of the powder mixture to a temperature above the melting point of the binder is from circulating hot liquid, hot air or steam, microwave or infrared sources, heating tape, high-shear mixing, rotating pan, extruder, fluidized bed granulator, or low shear mixing.

3. The method of claim 1 wherein the temperature is below the melting point of the BAS.

4. The method of claim 1 wherein the melting point of the binder is between 30° C. and 200° C.

5. The method of claim 1 wherein granules are formed from the mixture without subjecting the mixture to external pressure.

6. The method of claim 1 wherein the BAS is a non-compressible BAS.

7. The method of claim 6 wherein the BAS is selected from the group consisting of ibuprofen, acetaminophen, aspirin, naproxen, antibiotics, ketoprofen, indomethacin, ranitidine, sucralfate, vitamin C, probucol, nicotinic acid, aminocaproic acid, pentoxyfylline, quinidine gluconate, nifedipine, verapamil hydrochloride, cholestyramine, metoproplol tartrate, tocainamide hydrochloride, ethotoin, phenacemide, and carbidopa.

8. The method of claim 7 wherein the BAS is selected from the group consisting of ibuprofen, acetaminophen, and aspirin.

9. The method of claim 6 wherein the concentration of BAS in the granules is 80% or higher.

10. The method of claim 6 wherein the concentration of BAS in the granules is 85% or higher.

11. The method of claim 6 wherein the concentration of BAS in the granules is 90% or higher.

12. The method of claim 6 wherein the concentration of BAS in the granules is 95% or higher.

13. The method of claim 1 wherein the binder is polyethylene glycol.

14. The method of claim 1 wherein the binder is a waxy solid or semisolid.

15. The method of claim 14 wherein the binder is selected from the group consisting of stearic acid, poloxamers, glycerylesters of fatty acids, and hydrogenated vegetable oils.

16. The method of claim 1 which additionally comprises mixing with the BAS and the binder one or more excipients selected from the group consisting of filler, flavor, color, disintegrant, glidant, and lubricant.

17. A granule made by the method of claim 1.

18. A method for making tablets containing one or more biologically active substances (BAS) comprising mixing a binder with the BAS to obtain a powder mixture, heating the powder mixture to a temperature above the melting point of the binder to obtain an agglomerated powder, wherein the concentration of the binder in the powder mixture is at least that which will agglomerate the powder mixture when the binder is melted and is below that which will result in over-wetting of the powder mixture when the binder is melted, permitting granules to form from the agglomerated powder, and compressing the granules into tablets.

19. The method of claim 18 which additionally comprises mixing with the BAS and the binder one or more excipients selected from the group consisting of filler, flavor, color, disintegrant, glidant, and lubricant.

20. A method for making tablets containing one or more biologically active substances (BAS) comprising compressing a multiplicity of the granules produced by the method of claim 1 into tablets.

21. The method of claim 20 which additionally comprises mixing with the BAS and the binder one or more excipients selected from the group consisting of filler, flavor, color, disintegrant, glidant, and lubricant.

22. A tablet made by the method of claim 18.

23. A method for making capsules containing one or more biologically active substances (BAS) comprising mixing a binder with the BAS to obtain a powder mixture, heating the powder mixture to a temperature above the melting point of the binder to obtain an agglomerated powder, wherein the concentration of the binder in the powder mixture is at least that which will agglomerate the powder mixture when the binder is melted and is below that which will result in over-wetting of the powder mixture when the binder is melted, permitting granules to form from the agglomerated powder, and encapsulating the granules into capsules.

24. The method of claim 23 which additionally comprises mixing with the BAS and the binder one or more excipients selected from the group consisting of filler, flavor, color, disintegrant, glidant, and lubricant.

25. A method for making capsules containing one or more biologically active substances (BAS) comprising encapsulating a multiplicity of the granules produced by the method of claim 1 into capsules.

26. The method of claim 25 which additionally comprises mixing with the BAS and the binder one or more excipients selected from the group consisting of filler, flavor, color, disintegrant, glidant, and lubricant.

27. A capsule made by the method of claim 23.

28. A granule comprising one or more biologically active substances (BAS) and a binder, wherein the concentration of BAS in the granule is 80% or higher.

29. The granule of claim 28 wherein the concentration of BAS in the granule is 85% or higher.

30. The granule of claim 28 wherein the concentration of BAS in the granule is 90% or higher.

31. The granule of claim 28 wherein the concentration of BAS in the granule is 95% or higher.

32. The granule of claim 28 wherein the BAS is a non-compressible BAS.

33. The granule of claim 32 wherein the BAS is selected from the group consisting of ibuprofen, acetaminophen, aspirin, naproxen, antibiotics, ketoprofen, indomethacin, ranitidine, sucralfate, vitamin C, probucol, nicotinic acid, aminocaproic acid, pentoxyfylline, quinidine gluconate, nifedipine, verapamil hydrochloride, cholestyramine, metoproplol tartrate, tocainamide hydrochloride, ethotoin, phenacemide, and carbidopa.

34. The granule of claim 33 wherein the BAS is selected from the group consisting of ibuprofen, acetaminophen, and aspirin.

35. A tablet comprising one or more biologically active substances (BAS) and a binder, wherein the concentration of BAS in the tablet is 80% or higher.

36. The tablet of claim 35 wherein the concentration of BAS in the tablet is 85% or higher.

37. The tablet of claim 35 wherein the concentration of BAS in the tablet is 90% or higher.

38. The tablet of claim 35 wherein the concentration of BAS in the tablet is 95% or higher.

39. The tablet of claim 35 wherein the BAS is a non-compressible BAS.

40. The tablet of claim 37 wherein the BAS is selected from the group consisting of ibuprofen, acetaminophen, aspirin, naproxen, antibiotics, ketoprofen, indomethacin, ranitidine, sucralfate, vitamin C, probucol, nicotinic acid, aminocaproic acid, pentoxyfylline, quinidine gluconate, nifedipine, verapamil hydrochloride, cholestyramine, metoproplol tartrate, tocainamide hydrochloride, ethotoin, phenacemide, and carbidopa.

41. The tablet of claim 40 wherein the BAS is selected from the group consisting of ibuprofen, acetaminophen, and aspirin.
